SEQUENCE LISTING (10 sequences)

SEQ ID NO: 1 - DNA, 20 bp, Artificial Sequence
  Synthetic Construct
ccacgtcttcacatttggtg

SEQ ID NO: 2 - DNA, 20 bp, Artificial Sequence
  Synthetic Construct
agactgcgcctagtagttgt

SEQ ID NO: 3 - DNA, 24 bp, Artificial Sequence
  Synthetic Construct
atgttcgcctgaaacagagaccca

SEQ ID NO: 4 - DNA, 25 bp, Artificial Sequence
  Synthetic Construct
cttacagctggacttaaggcgtttc

SEQ ID NO: 5 - DNA, 20 bp, Artificial Sequence
  Synthetic Construct
accacagtccatgccatcac

SEQ ID NO: 6 - DNA, 21 bp, Artificial Sequence
  Synthetic Construct
tccaccaccctgttgcttgta

SEQ ID NO: 7 - DNA, 18 bp, Artificial Sequence
  Synthetic Construct
cccaaaggcttcttgttg

SEQ ID NO: 8 - DNA, 20 bp, Artificial Sequence
  Synthetic Construct
ctggtagttgttgtgagcat

SEQ ID NO: 9 - DNA, 21 bp, Artificial Sequence
  Synthetic Construct
atgagagccctcacactcctc

SEQ ID NO: 10 - DNA, 21 bp, Artificial Sequence
  Synthetic Construct
gccgtagaagcgccgataggc

(19) United States
(12) Patent Application Publication
(10) Pub. No.: US 2011/0301119 A1
(43) Pub. Date: Dec. 8, 2011

(54) PHARMACEUTICAL COMPOSITIONS CONTAINING FUCOIDAN FOR STIMULATING AND ACTIVATING OSTEOGENESIS

(75) Inventors: Se-Kwon Kim, Busan (KR); Young-Sook Cho, Busan (KR)

(73) Assignee: PUKYONG NATIONAL UNIVERSITY INDUSTRY-ACADEMIC COOPERATION FOUNDATION, BUSAN (KR)

(21) Appl. No.: 13/132,272

(22) PCT Filed: Nov. 5, 2009
(86) PCT No.: PCT/KR09/06476

§ 371 Date: Aug. 17, 2011

(30) Foreign Application Priority Data
Dec. 1, 2008 (KR) 10-2008-0120587

(51) Int. Cl. A61K 31/737; A61P 19/00; A61P 19/10; C07H 5/10

(52) U.S. Cl. 514/54; 536/122

(57) ABSTRACT

The present invention relates to pharmaceutical compositions containing fucoidan for stimulating and activating osteogenesis. The compositions according to the present invention contain fucoidan as an active ingredient to increase the activities of alkaline phosphatase and the production of osteocalcin, and to increase the production of bone morphogenetic protein (BMP-2).

### TECHNICAL FIELD

The present invention relates to a pharmaceutical composition comprising fucoidan. More particularly, the present invention relates to a pharmaceutical composition and a health function food for the stimulation and activation of osteoporosis, comprising fucoidan as an active ingredient which functions to increase the activity of alkaline phosphatase and the production of osteocalcin and to enhance the amount of bone morphogenetic protein-2 (BMP-2).

### BACKGROUND ART

Osteoporosis is a condition characterized by a reduction in skeletal mass resulting from an imbalance between bone resorption and bone formation, whereas bone homeostasis requires balanced interactions between oesteoblasts and osteoclasts (Ducy et al., 2000; Teitelbaum, 2000). Current drugs and functional foods used for bone health include bisphosphonates, calcitonin, estrogen, vitamin D analogs, ipriflavones, and isoflavones. These are all bone resorption inhibitors, which maintain bone mass by inhibiting the function of osteoclasts (Rodan and Martin, 2000). It is desirable, therefore, to have satisfactory bone-building (anabolic) agents that would stimulate new bone formation and correct the imbalance in the trabecular microarchitecture that is characteristic of established osteoporosis (Berg et al., 2003; Ducy et al., 2000).

Since new bone formation is the primary function of osteoblasts, agents for regulating bone formation act by either increasing the proliferation of cells of the osteoblastic lineage or inducing differentiation of the osteoblasts (Ducy et al., 2000). Recent studies have suggested the beneficial effects of sulfated monosaccharides (SGlc) and polysaccharides (anionic PEC) on osteoblastic cell proliferation and bone regeneration (Kim et al., 2007; Naghata et al., 2005).

Brown seaweeds are known to produce different polysaccharides, namely alginates, laminarans and fucoidans (Painter, 1983; Percival, 1967). Of them, fucoidans are extracted predominantly from sea mustard (U. pinnatifida), and Laminaria japonica, which are commercially available in East Asian countries including Korea, China and Japan. Fucoidans, a class of sulfated polysaccharides, usually contain large proportions of L-fucose and sulfate, together with minor amounts of other sugars e.g, xylose, galactose, mannose and glucuronic acid (Duarte et al., 2001; Percival, 1967). Fucoidans have recently been reported to have physiological activities including antioxidative, anticoagulant, anti-thrombotic, anti-inflammatory, anti-tumoral and anti-viral activity (Chevolot et al., 2001; Cumashi et al., 2007; Mourao, 2004; Itoh et al/, 1993; Maruyama et al., 2006; Thompson and Dragar, 2004). Nowhere have the beneficial effects of fucoidans on bone health and formation been disclosed in the previous literature and reports.

Leading to the present invention, intensive and thorough research into the ability of fucoidans to induce the cell differentiation of the osteosarcoma cell line MG-63, conducted by the present inventors, resulted in the finding that fucoidan extracted from the marine brown alga Undaria pinnatifida can increase the activity of alkaline phosphatase (ALP) and the level of osteocalcin (OC) which are phenotypic markers for early-stage osteoblastic differentiation and terminal osteoblastic differentiation, respectively, and that fucoidan has positive effects on bone morphogenic protein-2 (BMP-2) which is an important factor in bone formation, remodeling and mineralization. This research provides new insight into the osteoblastic differentiation of fucoidan and the possibility for its application in bone health supplements.

### DISCLOSURE

### Technical Problem

It is therefore an object of the present invention to provide the use of fucoidan as an osteogenesis stimulator.

It is another object of the present invention to provide a pharmaceutical composition for the stimulation and activation of osteogenesis comprising fucoidan as an active ingredient.

It is a further object of the present invention to provide a health functional food for stimulating and activating osteogenesis, comprising fucoidan as an active ingredient.

### Technical Solution

The above-mentioned objects of the present invention were achieved by various analytical experiments including the treatment of the human osteosarcoma cell line MG-63 with fucoidan extracted from marine brown alga, cell viability assays, an alkaline phosphatase (ALP) activity assay, mineralization analysis, osteocalcin and BMP-2 level analysis and RT-PCR analysis, which demonstrated that fucoidan can increase the activity of alkaline phosphatase and the production of osteocalcin and enhance the production of bone morphogenetic protein-2 (BMP-2).

In accordance with an aspect thereof, the present invention provides a pharmaceutical composition for the stimulation and activation of osteogenesis, comprising fucoidan as an active ingredient.

Having the activity of stimulating and activating osteogenesis, the pharmaceutical composition comprising fucoidan according to the present invention is useful for the treatment and prevention of bone-related disorders such as osteoporosis, bone fractures, etc.

As used herein, the term “active ingredient” means a material or a material group (inclusive of herb medicines of which the pharmaceutical principle is unknown) that is anticipated to express the efficacy and effect of the medicine thanks to its intrinsic pharmacological action.

The pharmaceutical composition comprising fucoidan according to the present invention may further comprise suitable vehicles, excipients and diluents.

For administration, the active ingredient of the present invention may be used in the form of pharmaceutically acceptable salts. In addition, the active ingredient of the composition may be administered alone or in association with other pharmaceutically active compounds or may be used in the form of an appropriate assembly.

In accordance with another aspect thereof, the present invention provides a health functional food for stimulating and activating osteogenesis, comprising fucoidan as an active ingredient.

The term “health functional food,” as used herein, is intended to refer to a food prepared or processed from raw materials or components which have functionality beneficial to the body as stipulated by Korean Law 6727 regarding health functional foods. Herein, the term “functionality” means the ability to regulate nutrients according to the structure or function of the body or to provide such effects beneficial for the health as physiological activities upon the uptake of food.

The health functional food for stimulating and activating osteogenesis in accordance with the present invention comprises the extract in an amount of from 0.01 to 95% by weight based on the total weight of the composition, and preferably in an amount of from 1 to 80% by weight.

In addition, with the aim of stimulating and activating osteogenesis, the health functional food may be formulated or processed into various forms such as tablets, powders, granules, liquids, pills, and so on.

In accordance with the present invention, fucoidan can bring about a significant increase in the activity of alkaline phosphatase and the production of osteocalcin in various stages of osteoblastic differentiation. Also, fucoidan can promote bone mineralization which is responsible for the activation of bone morphogenetic protein (BMP-2). Disclosing the ability of fucoidan to promote the early stages of osteoblastic differentiation, the present invention could provide new insight into the osteoblastic differentiation of fucoidan and the possibility for its application in bone health supplements.

### Advantageous Effects

As described above, the pharmaceutical composition for osteogenesis comprising fucoidan as an active ingredient in accordance with the present invention can promote the activity of alkaline phosphatase and increase the levels of osteocalcin and bone morphogenetic protein (BMP-2).

### DESCRIPTION OF DRAWINGS

FIG. 1 is a graph showing effects of fucoidan on the viability of MG-63 cells in a dose-dependant manner.

FIG. 2 is a graph showing effects of fucoidan (100 μg/ml) on the viability of MG-63 cells in a time-dependant manner.

FIG. 3 shows results of an alkaline phosphatase assay performed on the MG-63 cells treated with various concentrations of fucoidan for 48 hours.

FIG. 4 shows effects of fucoidan on the osteocalcin secretion of MG-63 cell.

FIG. 5 shows representative cell cultures after they were stained with Alizarine Red-S for 7 days.

FIG. 6. shows the effect of fucoidan on the production of BMP-2.

### MODE FOR INVENTION

A better understanding of the present invention may be obtained through the following examples which are set forth to illustrate, but are not to be construed as limiting the present invention.

### EXAMPLES

Fucoidan (total polysaccharides: 62.12% and sulfate: 34.20%) extracted from brown alga (Undaria pinnatifida) was purchased from Haewon Biotech, Inc. Seoul, Korea. FBS (fetal bovine serum), MEM (minimal essential medium), penicillin G, and streptomycin were purchased from GIBCO-BRL; Gaithersburg, Md., USA. A BMP-2 ELISA was obtained from R&D Systems, Minneapolis, Minn., USA. An osteocalcin ELISA kit was purchased from Takara; Tokyo, Japan and p-nitrophenyl phosphate and MIT [3-(4,5-dimethylthiazol-2-yl)-2,5-diphenyltetrazolium bromide] were purchased from Sigma; St. Louis, Mo. RT-PCR reagents were purchased from Promega; Madison, Wis., USA.

All data was expressed as mean±S.D. Statistical comparisons of the results were made using analysis of variance (ANOVA). Significant differences (P<0.05) between the means of the control and test groups were analyzed by Dunnett's test.

### Example 1

### Cell Culture

The human osteosarcoma cell line MG-63 (CRL-1427) was obtained from the ATCC (American Type Culture Collection). MG-63 cells were cultured in MEM media supplemented with 10% FBS and antibiotics (100 IU/mL penicillin G and 100 μg/mL streptomycin).

### Example 2

### Cell Viability Assay

MTT was used as an indicator of cell viability as determined by mitochondrial-dependent reduction to formazan. In brief, the cells were seeded and then treated with various reagents at the indicated time intervals. After various treatments the medium, the medium is removed and the cells were incubated with a solution of 0.5 mg/mL MTT. After incubation for 3 hours at 37° C. and 5% CO₂ atmosphere, the supernatant was removed and the formation of formazan was observed by monitoring signals using a microplate reader.

### Example 3

### Alkaline Phosphatase (ALP) Activity Assay

Cells were seeded at a density of 5×10³ cells/well into 96-well plates and incubated for 24 hours. The addition of a test reagent to the wells was followed by incubation for 2 days. Subsequently, the cells were washed three times with saline and the cellular protein concentration was determined by incubation in B (bicinchoninic acid) protein assay reagent at 37° C. for 1 hour. The reaction was terminated with 1 M NaOH, followed by the measurement of absorbance at 560 nm.

Alkaline phosphatase activity in the cells was assayed after appropriate treatment periods by washing cells three times with physiological saline and then measuring cellular activity after incubation for 1 hour at 37° C. in 0.1 M NaHCO₃—Na₂CO₃ buffer, pH 10, containing 0.1% Triton X-100, 1.5 mM MgCl₂₂ and 15 mM p-nitrophenyl phosphate. The reaction was stopped by adding 1 M NaOH before measuring absorbance at 405 nm. A unit of phosphatase activity is defined as the amount of enzyme activity that will liberate 1 μm of p-nitrophenol per hour (Eichner et al., 2002).

### Example 4

### Analysis of Mineralization

After treatment for 7 days in 24-well plates, the cells were analyzed for their degree of mineralization using Alizarin Red (Alizarin Red; Sigma Chemical, St Louis, Mo., USA) staining. In brief, cells were fixed for 1 hour with 70% (v/v) ethanol and then were stained with 40 mM Alizarin Red S in deionized water (pH=4.2) for 1 hour at room temperature. After removal of Alizarin Red S solution by aspiration, the cells were incubated in PBS for 15 min at room temperature on an orbital rotator. Thereafter, the cells were washed once with PBS and destained for 15 min with 10% (w/v) cetylpyridinium chloride in 10 mM sodium phosphate (pH=7.0). The extracted stain was then transferred into 96-well plates and absorbance at 562 nm was measured using a microplate reader.

### Example 5

### Quantitative Assay of Osteocalcin and BMP-2

Osteocalcin and BMP-2 ELISA kits were used to detect osteocalcin and BMP-2 levels, respectively. In brief, cells were treated with various concentrations of fucoidan. The culture media were harvested and analyzed for the level of osteocalcin and BMP-2. These samples were placed in 96-well microliter plates coated with monoclonal detective antibodies and incubated for 2 hours at room temperature. After removing unbound material with washing buffer (50 mM Tris, 200 mM NaCl, and 0.2% Tween 20), horseradish peroxidase conjugated streptavidin was added to bind to the antibodies. Horseradish peroxidase catalyzed the conversion of a chromogenic substrate (tetramethylbenzidine) to a coloured solution, with colour intensity proportional to the amount of protein present in the sample. The absorbance of each well was measured at 450 nm. Results took the form of the percentage of change in activity compared to the untreated control (Kuo et al., 2005).

### Example 6

### RT-PCR Analysis

Expression levels of BMP-2 mRNA were examined using RT-PCR. RT-PCR was also performed with GAPDH used independently as an internal control. RNA was isolated with TRIzol reagent Aliquots (2 μg) of total RNA were reverse transcribed to cDNA using AMV reverse transcriptase. Oligonucleotides for use in PCR were as follows: 5′-CCACGTCTTCACATTTGGTG-3′(forward primer) and 5′-AGACTGCGCCTAGTAGTTGT-3′ (reverse primer) for human ALP mRNA; 5′-ATGTTCGCCTGAAACAGAGACCCA-3′ (forward primer) and 5′-CTTACAGCTGGACTTAAGGCGTTTC-3′ (reverse primer) for human BMP-2 mRNA; 5′-ACCACAGTCCATGCCATCAC-3′ (forward primer) and 5′-TCCACCACCCTGTTGCTTGTA-3′ (reverse primer) for human GAPDH; and 5′-CCCAAAGGCTTCTTCttGTTG-3′ and 5-CTGGTAGTTGTTGTGAGCAT-3′, and 5′-ATGAGAGCCCTCACACTCCTC-3′ (forward primer) and 5′GCCGTAGAAGCGCCGATAGGC (reverse primer) for osteocalcin (Drissi et al, 1997). The PCR products were detected by 1.5% agarose gel electrophoresis and photographed.

Examples 1 to 6 revealed the following results.

Fucoidan is one of the sulfated polysaccharides derived from marine brown alga. Sulfated polysaccharides, such as glucosamine sulfate, chitosan sulfate, heparin and heparan sulfate, are macromolecules associated with cell surfaces and extracellular matrices (Xiao et al., 2004; Naghata et al, 2005). These polysaccharides have been shown to interact directly with a number of growth factors including BMPs, via highly negatively charged polysaccharide chains. Indeed, heparin-affinity chromatography was used to purify the BMP activity from extracts prepared by employing demineralized bone matrix (Wang et al., 1990).

To clearly understand the effect of fucoidan on bone health and mineralization, as described above, fucoidan extracted from marine brown alga were analyzed for its ability to increase the activity of alkaline phosphatase (ALP), a phenotypic marker for early stage osteoblastic differentiation, and the level of osteocalcin (OC), a phenotypic marker for terminal stage osteoblastic differentiation. Also, the positive effect of fucoidan on the production of bone morphogenetic protein-2 (BMP-2), an important factor in osteogenesis and mineralization, was studied.

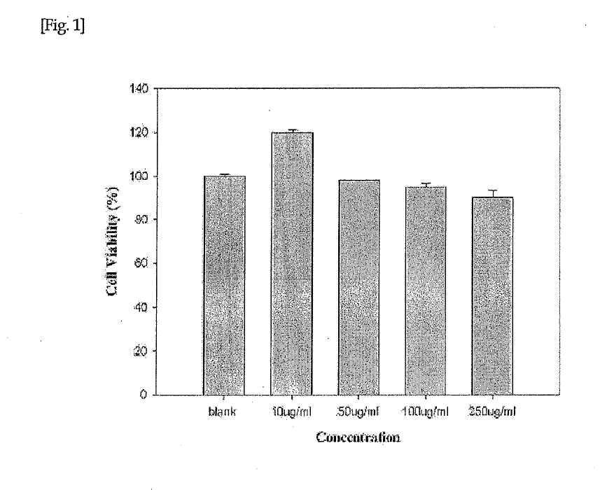

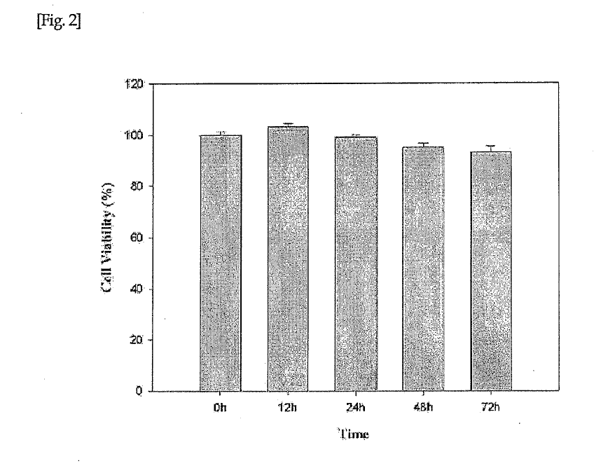

First, the effect of fucoidan on the cell growth of MG-63 was measured at various concentrations by MTT assay. As a result, fucoidan was observed to have no significant influences on cell viability at the concentrations used (10 g/mL-250 g/mL) after 24 hours of treatment for the cell line (FIG. 1). In addition, treatment with 100 g/mL of fucoidan did not exhibit significant effects on cell viability for 72 hours (FIG. 2).

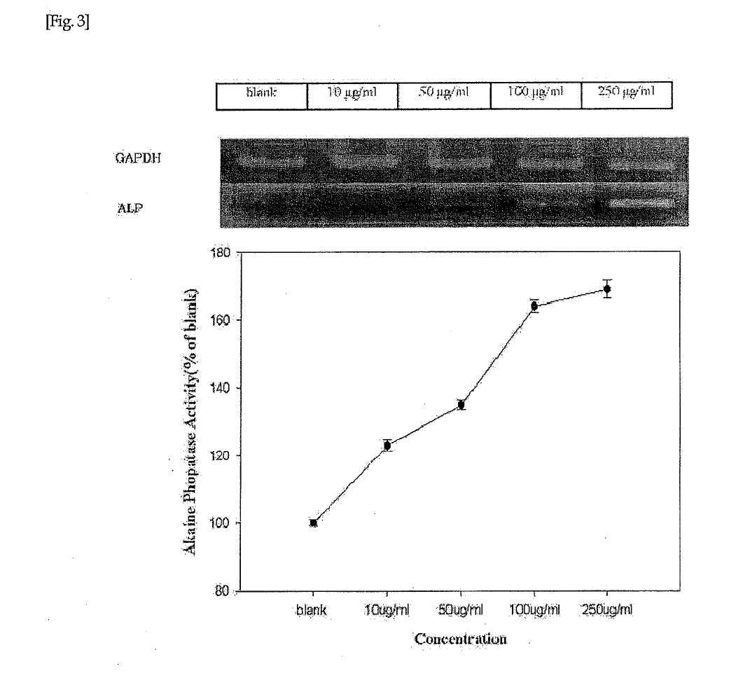

The effect of fucoidan on the maturation of osteoblasts was studied by determining alkaline phosphatase activity in MG-63 cells. The results showed that fucoidan increased alkaline phosphatase activity and mRNA expression in a dose-dependent manner after 48 hour of treatment in the cell (FIG. 3).

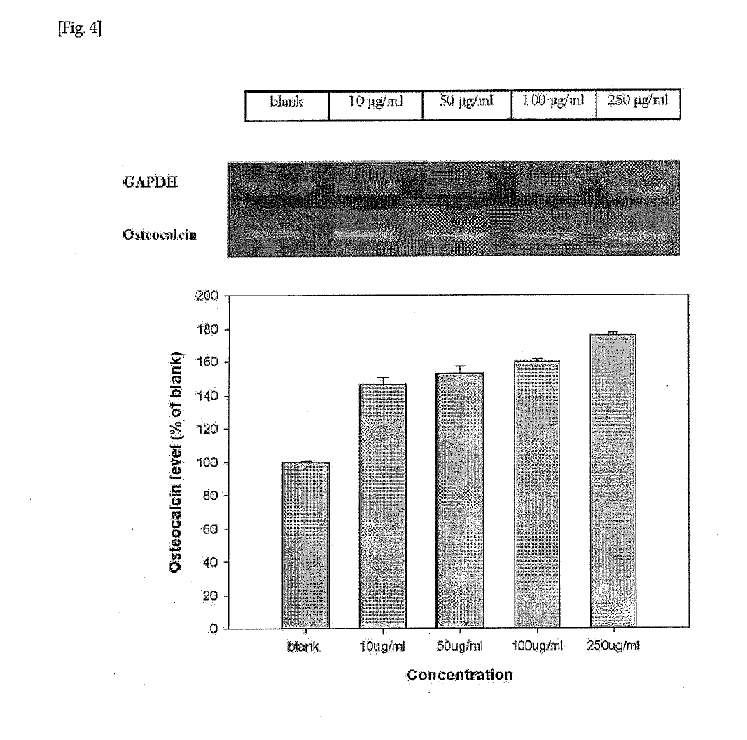

The effect of fucoidan on terminal differentiation of osteoblast-like cells was also studied by determining the concentrations of osteoblast in the culture media. Osteocalcin is a specific cell marker protein for the terminal cell differentiation of osteoblasts which is secreted into a cell culture media by the osteoblast MG-63 treated for 48 hours with fucoidan was observed to undergo a significant increase in osteocalcin synthesis. Further, fucoidan stimulated osteocalcin synthesis in a concentration-dependent manner (FIG. 4).

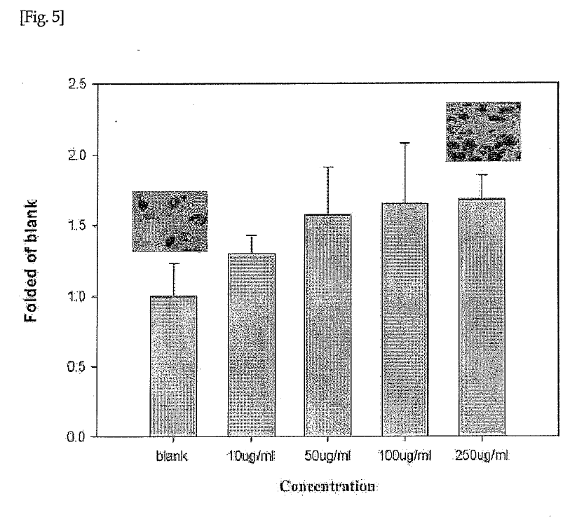

According to Alizarin Red S staining data (FIG. 5), fucoidan increased the amounts of hydroapatite in the cell. The mineral intercellular level was higher in a concentration-dependent manner.

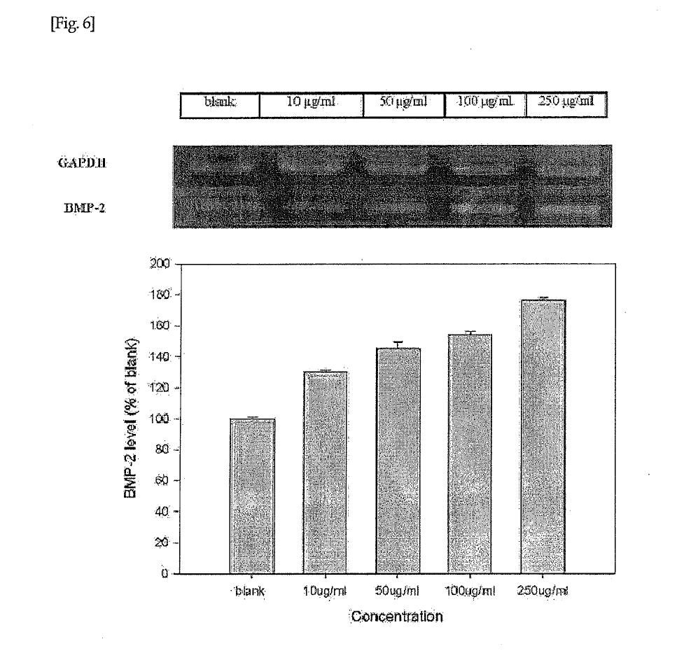

An examination was made of the expression of the BMP gene using RT-PCR and of the synthesis of bone morphogenetic proteins in the presence or absence of fucoidan using BMP ET ISA kits. As a result, fucoidan induced a significant increase in BMP-2 mRNA and protein synthesis in MG-63 cells. The production of BMP-2 was significantly increased in a dose-dependent manner after 24 hours from treatment with fucoidan (FIG. 6). In addition, the expression of BMP-2 gene in the presence or absence of fucoidan in MG-63 cells was examined using RT-PCR.

Bone morphogenetic proteins (BMP) play an important role in the process of bone formation and remodeling (Sykaras and Opperman, 2003). The stimulation of osteoblastic differentiation is characterized mainly by an increased expression of alkaline phosphatase and osteocalcin (Xiao et al, 2004). Particularly, recombinant human bone morphogenetic protein-2 (rhBMP-2) is known to play an important role in osteogenesis and enhance the healing of fractures. In fracture healing, there is increased BMP receptor expression in osteogenic cell near the fracture, in fibroblast-like spindle ells, and in fibroblasts involved in endochondral ossification (Onishi T, et al., 1998). Interestingly, the promotion of bone-healing benefits by BMP-2 is believed to be due to their ability to stimulate the proliferation and differentiation of mesenchymal and osteoprogenitor cells, and both are angiogenic. Bone morphogenetic proteins (BMPs) are members of the TFG-β superfamily, which regulate embryonic development and also induce ectopic bone formation in developed tissues (Urist, 1965; Hogan, 1996; Hollevilleet at, 2003). In cells of the osteoblast lineage, BMPs 2, 4, and 7 induce the expression of alkaline phosphatase, type I collagen, and other non-collagenous bone proteins found in osteoids, a phenotype consistent with differentiated osteoblasts (Cheifetz et at, 1996; Li et at, 1996; Cheng et at, 2003).

1. A pharmaceutical composition for stimulation and activation of osteogenesis, comprising fucoidan as an active ingredient.

2. The pharmaceutical composition of claim 1, wherein the composition is effective in preventing and treating osteoporosis and bone fracture disorders by stimulating and activating osteogenesis.

3. A health functional food for stimulating and activating osteogenesis, comprising fucoidan as an active ingredient.

4. The health functional food of claim 3, wherein the food is effective in preventing osteoporosis and bone fracture disorders by stimulating and activating osteogenesis.
